(19) United States
(12) Patent Application Publication
(10) Pub. No.: US 2021/0145993 A1
(43) Pub. Date: May 20, 2021

(54) CONTAINER TREATMENT PLANT

(71) Applicant: KRONES AG, Neutraubling (DE)

(72) Inventors: Tim HUFENBACH, Klein Rheide (DE); Jan MUENZER, Harrislee (DE); Bernhard UNRECHT, Neutraubling (DE); Merret SACHT, Langballig (DE)

(21) Appl. No.: 17/045,156

(22) PCT Filed: Mar. 14, 2019
(86) PCT No.: PCT/EP2019/056386

§ 371 Date: Oct. 2, 2020

(30) Foreign Application Priority Data
Apr. 4, 2018 (DE) 10 2018 205 009.0

(51) Int. Cl. A61L 2/18; C01B 11/02; A61L 2/04

(52) U.S. Cl. CPC A61L 2/18; A61L 2101/06; A61L 2/04; C01B 11/024

(57) ABSTRACT

Container treatment plant for treating containers, such as bottles, in the beverage processing industry comprising a chlorine dioxide producer, a chlorine dioxide consumer, comprising at least one container treatment machine, in particular a pasteurizer, such as a tunnel pasteurizer, and a metering device that can introduce a solution containing chlorine dioxide produced in the chlorine dioxide producer into the chlorine dioxide consumer, where the chlorine dioxide producer is configured to produce chlorine dioxide through a reaction of sodium chlorite and sulfuric acid.

The present invention relates to a container treatment plant for treating containers, such as bottles, in the beverage processing industry according to claim 1 and a method for disinfecting process water and/or components of a container treatment plant according to claim 7.

### PRIOR ART

Container treatment plants with one or more container treatment machines and possibly additional components, such as storage containers or the like, are already employed in the beverage processing industry. Such container treatment plants can be constructed as “blocked” machine groups in which the containers pass through the container treatment machines consecutively and are treated in the machines.

The container treatment machines include not only those machines that allow the containers to be processed and, for example, to equip the containers with certain features, such as labels or printed images, but also equipment, such as fillers machines and pasteurizers. Biological material can deposit in particular in the case of pasteurizers that pasteurize a filled container and biofilms can form in the container treatment machine, e.g. due to the product used, but also microbiotic residues in the process water that is used in the machine can form. Once such a biofilm has formed in the machine, it is often difficult to be removed.

In order to avoid the permanent formation of biofilms in the container treatment machine, disinfectants, in particular solutions containing chlorine dioxide, are used. Corresponding devices and methods are known, for example, from DE 10 2012 109 758 B3.

However, the known methods have the drawback that the disinfection agents used or their by-products have a highly corrosive effect and can therefore damage the container treatment machine and also individual bottles and, in this case, crown corks in particular. This property also arises with chlorine dioxide when it is produced using hydrochloric acid, since the residues of the starting materials in the solution produced can cause corrosion of materials.

### OBJECT

Starting out from known prior art, the technical object to be satisfied is therefore to ensure reliable disinfection and the prevention of biofilms in container treatment plants while corrosion of individual components is avoided to the extent possible.

### SOLUTION

This object is satisfied according to the invention with the container treatment plant according to claim 1 and the method according to claim 7. Advantageous developments of the invention are comprised in the dependent claims.

The container treatment plant according to the invention for treating containers, such as bottles, in the beverage processing industry comprises a chlorine dioxide producer, a chlorine dioxide consumer comprising at least one container treatment machine, in particular a pasteurizer, such as a tunnel pasteurizer, and a metering device that can introduce a solution containing chlorine dioxide produced in the chlorine dioxide producer into the chlorine dioxide consumer, where the chlorine dioxide producer is configured to produce chlorine dioxide through a reaction of sodium chlorite and sulfuric acid.

The chlorine dioxide producer is to be understood as being any device which is configured in such a way that the reaction according to the invention of chlorine dioxide from sodium chlorite and sulfuric acid can be carried out (in an aqueous solution). This includes, in particular, chemical reactors that can maintain certain temperatures and pressures over a long period of time that are considered particularly suitable for the production. The chlorine dioxide producer can also comprise suitable storage containers for the sulfuric acid and the sodium chlorite in an aqueous solution or also in high concentration. Furthermore, the chlorine dioxide producer can comprise or be associated with a water supply so that the reaction of sodium chlorite and sulfuric acid to form chlorine dioxide can take place in an aqueous solution and a solution containing chlorine dioxide can be produced which has a desired or predetermined concentration.

The chlorine dioxide consumer is generally a device (presently container treatment machine) that uses chlorine dioxide to perform certain functions. They preferably include disinfection processes in pasteurizers or the like.

The metering device is a device which is suitable to feed a quantity of solution containing chlorine dioxide to a chlorine dioxide consumer. The feeding is there not restricted, but can comprise atomization, continuous feeding or feeding taking place only at time intervals.

In addition to chlorine dioxide and water, the chlorine dioxide solution also contains the reaction products that are formed in addition to chlorine dioxide when sodium chlorite and sulfuric acid react to form chlorine dioxide. They are in particular sodium chloride and sodium sulfate.

The residues of sodium chloride and sodium sulfate have a significantly less corrosive effect on the metal components typically used in container treatment plants, while the advantageous disinfecting properties of the chlorine dioxide from the solution can be used to effectively prevent the formation of biofilms and also microbiotic deposits or to dissolve them if necessary. What adds to this is that the solutions with sodium chloride and sodium sulphate used are harmless and that contact with food therefore does not directly lead to contamination and therefore rejects.

In one embodiment, the chlorine dioxide consumer comprises a cooling tower and/or a tunnel heat exchanger and/or a tunnel heater. While the chlorine dioxide can also be used for disinfection at other locations in the container treatment plant, it can be employed in particular in cooling towers and tunnel heat exchangers, since the disinfection of the water used or the like can prevail a favorable environment for the formation of biofilms.

Furthermore, it can be provided that the container treatment plant comprises a buffer tank for receiving the solution produced in the chlorine dioxide producer and for delivering the solution received to the metering device. The introduction of the chlorine dioxide or the solution, respectively, into the chlorine dioxide consumer by the metering device can therefore be decoupled or substantially decoupled from the production of the solution in the chlorine dioxide producer, which also allows for the solution to not be introduced into the chlorine dioxide consumer continuously, while the chlorine dioxide producer produces the solution containing chlorine dioxide, for example, continuously.

Furthermore, it can be provided that a delivery rate of the metering device can be controlled by a control unit. The delivery rate is there given as the quantity of solution or the quantity of chlorine dioxide that is delivered per unit of time (for example per hour, minute or second). Controlling this delivery rate with the control unit is to be understood as meaning that the delivery rate of the metering device is set with the aid of the control unit and the control unit can cause the metering device to deliver the respective solution at the delivery rate selected. It is then possible to adjust the delivered quantity of solution to certain factors, such as the degree of contamination in the chlorine dioxide consumer.

In one embodiment, the metering device can introduce the solution into a wet region of the chlorine dioxide consumer. A wet region is a region in the chlorine dioxide consumer in which water or other liquid substances come into contact with the containers or components of the chlorine dioxide consumer outside a closed circuit. This includes, for example, cleaning baths, but also regions in which the containers are sprayed or acted upon with liquids, such as water, in order to achieve, for example, cooling or slow heating, like in a pasteurizer.

It is provided in a further development of this embodiment that the container treatment plant comprises a pH value sensor for measuring the pH value in the wet region and/or a chlorine dioxide sensor (such as a kind of a redox sensor) for measuring a concentration of chlorine dioxide in the wet region. The pH value and/or the concentration or the absolute quantity of chlorine dioxide can be determined with the aid of these sensors preferably in real time by continuous measurement or by measurement within short time intervals of one second, a few seconds or minutes in order to be able to detect adverse effects of the use of chlorine dioxide or an acidic or basic environment in the chlorine dioxide consumer.

The method according to the invention for disinfecting process water and/or a component of a container treatment plant comprising a chlorine dioxide producer, a chlorine dioxide consumer comprising at least one container treatment machine, in particular a pasteurizer, such as a tunnel pasteurizer, and a metering device comprises that the metering device introduces solution containing chlorine dioxide into the chlorine dioxide consumer so that process water in the chlorine dioxide consumer and/or a component of the chlorine dioxide consumer is acted upon with the solution, where the solution is produced in the chlorine dioxide producer through a reaction of sodium chlorite and sulfuric acid.

Process water is presently to be understood as being the liquid (typically water) that is used in the container treatment machine, for example, to heat or clean containers. This does not presently need to be pure water. Aqueous solutions that contain other substances in addition to water are also presently conceivable and are summarized under the term “process water”.

Reliable disinfection of process water used in the machine as well as components within a chlorine dioxide consumer is made possible with this method without the negative corrosive properties of commonly used disinfectant solutions leading to premature undesirable wear.

The method can comprise that the introduction of the solution by the metering device is controlled with the aid of a control unit so that the introduction takes place continuously or as surge dosing.

Surge dosing comprises the introduction of the solution over a short period of time (a few seconds, such as, for example, 1 second, 2 seconds or 10 seconds), but in large quantities, so that there is an abrupt increase in the concentration of chlorine dioxide in the chlorine dioxide consumer and in in particular in an aqueous solution used in the chlorine dioxide consumer. In contrast, continuous introduction of solution realizes a slow increase in the concentration or, due to further reactions and the exchange of aqueous solution, a uniform concentration of chlorine dioxide.

Surge dosing can be advantageous if considerable contamination of the chlorine dioxide consumer is given due to a malfunction, whereas a continuous supply can basically prevent or at least delay the formation of biofilms.

It can be provided that the pH value is measured in a wet region of the chlorine dioxide consumer and/or the concentration of chlorine dioxide in the chlorine dioxide consumer is measured and the introduction of the solution into the wet region is controlled ion dependence of the pH value and/or the concentration. If the objective is to maintain a certain pH value or a certain concentration of chlorine dioxide, then this method can reliably maintain the desired value even over longer periods of operation of the container treatment plant.

It can also be provided that the chlorine dioxide consumer comprises a pasteurizer with at least one heating zone, a pasteurization zone, and a cooling zone, where the metering device feeds the solution to the pasteurizer as follows:

in all zones at the same or different concentrations; or

only in the heating zone or only in the cooling zone or only in the pasteurization zone; and/or

in at least one zone during a standstill of the pasteurizer; and/or

in a measured value-controlled or throughput-controlled manner.

The heating zone and the cooling zone are regions of the pasteurizer in which the containers fed are heated or cooled down. They can be connected to one another via a common water circuit (recuperation circuit) in such a way that the heat from the containers absorbed by the cooling medium (in particular water) in the cooling zone is used to heat up the containers in the heating zone. The pasteurization zone, which is arranged in the direction of transport of the containers between the heating zone and the cooling zone, is a region in which the containers and possibly products contained therein are heated to the pasteurization temperature and held at this temperature for a certain period of time (a few minutes to hours) to realize disinfection.

It can also be provided that the concentration of chlorine dioxide in the solution produced by the chlorine dioxide producer is 15,000 to 25,000 ppm, in particular 17500 to 22,500 ppm, and/or the concentration of the solution that is introduced into the chlorine dioxide consumer by the metering device is diluted in a first dilution stage to a concentration of 2000 to 4000 ppm, preferably 2500 to 3500 ppm, particularly preferably 3000 ppm, and/or the concentration of the solution that is introduced into the chlorine dioxide consumer by the metering device is diluted in a second dilution stage to 10 up to 1000 ppm, preferably 50 to 300 ppm, particularly preferably 200 ppm, and/or a chlorine dioxide concentration in the process water of the consumer is specified at 0.01 to 10 ppm, preferably 1 to 5 ppm, particularly preferably 1.5 ppm, as the target value. In this context, the first dilution stage is understood to mean the state of the solution containing chlorine dioxide which is reached when the solution produced in the chlorine dioxide producer is first diluted. The second dilution stage is therefore the state of further dilution that is reached after dilution of the first dilution stage. Due to the decomposition of chlorine dioxide already during the transport of the solution into the chlorine dioxide consumer and also during the flow through the chlorine dioxide consumer, a measured value of the concentration of chlorine dioxide in the process water can differ from the desired target value.

Holding available a solution with as high a concentration as possible reduces the total quantity of solution to be held available because less water has to be added to it. Using a solution with a relatively low concentration of chlorine dioxide in the chlorine dioxide consumers can minimize the negative effects of the chlorine dioxide, sodium chloride, or sodium sulfate on components or containers.

It can be provided that the chlorine dioxide producer continuously produces the solution through the reaction of sodium chlorite with sulfuric acid and feeds the solution produced to a buffer tank, where the metering device removes the solution from the buffer tank. Decoupling the production and the distribution of the solution can thus be achieved.

In a further development of this embodiment, the metering device feeds the solution to several chlorine dioxide consumers, where the metering device sets the concentration of chlorine dioxide in the solution for each chlorine dioxide consumer independently by adding water to the solution before the metering device introduces the solution into the consumer. Since the solution produced by the chlorine dioxide producer has a certain concentration, a “second” solution having a lower concentration can be produced by adding water in a selective manner, where the concentration can be adjusted in dependence of the needs of the respective chlorine dioxide consumer.

It can further be provided that the solution is introduced into the chlorine dioxide consumer at a temperature T<50° C., at least in the region of the consumer into which the solution is introduced. In this way, unintentional decomposition of the chlorine dioxide in the solution and the associated deterioration in the disinfection effects can be avoided.

### BRIEF DESCRIPTION OF THE FIGURES

FIG. 1 shows schematically a container treatment plant according to one embodiment; and

FIG. 2 shows a pasteurizer in combination with a metering device according to one embodiment.

### DETAILED DESCRIPTION

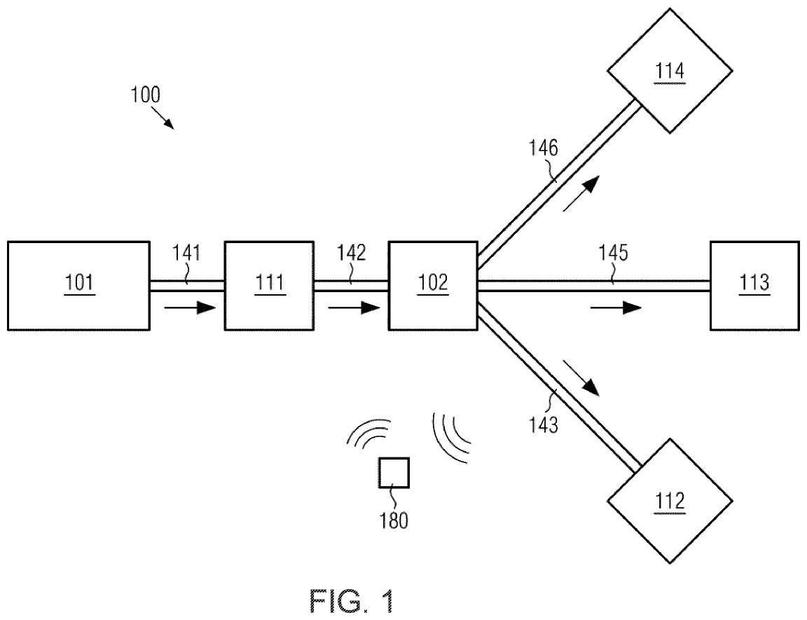

FIG. 1 shows schematically a container treatment plant 100 for treating containers such as bottles. This container treatment plant can comprise one or more container treatment machines 112 to 114, as they are already well known. Container treatment machines 112 to 114 can therefore be, for example, machines for manufacturing glass bottles or bottles made of plastic, such as PET. The provision of a pasteurizer, in particular a tunnel pasteurizer, is particularly preferred. Furthermore, the container treatment machines can comprise bottle cleaning machines that can clean recyclable bottles with water and cleaning agents. In addition, machines can be employed that fill the bottles with a product and close them (filler, capper) and then provide them with features such as labels or printed images. It can be provided in connection with fillers, in particular in the case of perishable products, that pasteurization takes place in the direction of transport of the bottles through the container treatment plant downstream of the filler with the aid of the pasteurizer already mentioned.

According to the invention, at least one of the container treatment machines is configured as a chlorine dioxide consumer. This can be, for example, machine 112. A chlorine dioxide consumer is to be understood to be such container treatment machine that requires chlorine dioxide or a solution containing chlorine dioxide either continuously or at time intervals (periodically or non-periodically). This includes in particular machines in which biofilms and microbiotic deposits can form. Machines can there be considered in which a product is filled into containers or closed containers filled with product are processed. This includes in particular fillers and pasteurizers as well as heat exchangers and heaters. Cooling towers which can be used to cool container treatment plants can also be treated with a respective solution containing chlorine dioxide in order to, for example, suppress the formation of biofilms.

Due to product deposits in the region of such container treatment machines, bacteria can settle on these deposits over a long period of operation and lead to the formation of biofilms that are difficult to remove. This occurs in particular in regions of a pasteurizer in which the temperatures are not high enough to reliably kill germs, in particular bacteria. This includes, for example, the heating and cooling region of pasteurizers or the regions of a cooling tower that come into contact with water.

It is provided according to the invention that a or each chlorine dioxide consumer (i.e. for example, one or more of container treatment machines 112 to 114) is connected to a metering device 102, possibly via respective transport systems for a solution 143 to 146 containing chlorine dioxide, so that metering device 102 can feed each chlorine dioxide consumer the solution containing chlorine dioxide. The solution containing chlorine dioxide is preferably fed into a wet region of the chlorine dioxide consumer, i.e. into a region which is filled with water or is at least acted upon therewith.

Furthermore, it is provided according to the invention that metering device 102 is connected to a chlorine dioxide producer 101 via a suitable line. This can be implemented, for example, by an end-to-end line 141 to 142. In this case, element 111 described later is not provided and chlorine dioxide producer 101 is therefore connected directly to metering device 102.

The metering device can be understood in the broadest sense as being a device which is configured to feed or introduce a certain quantity of solution containing chlorine dioxide into the chlorine dioxide consumers. While feed lines 143 to 146 are shown separately, they can also be understood to be part of metering device 102 which can then extend all the way into the chlorine dioxide consumer. Inside the chlorine dioxide consumer, the metering device can be connected to suitable devices for feeding the solution containing chlorine dioxide. These can be, for example, pumps which introduce the solution containing chlorine dioxide into a continuous flow of liquid (water or the like) which then circulates in some manner within the chlorine dioxide consumer.

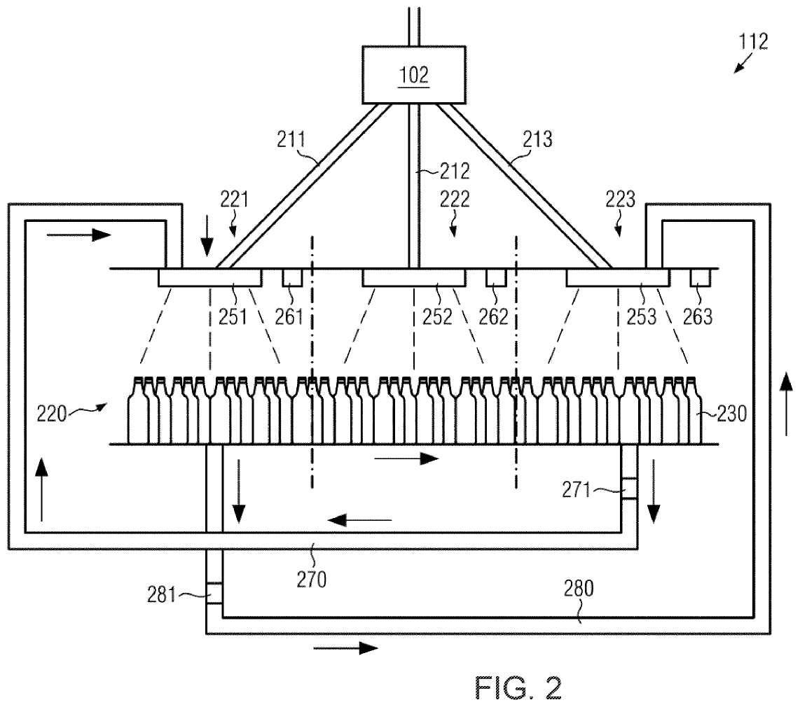

The solution containing chlorine dioxide is preferably introduced into wet regions, as described above, since contact with the product occurs largely there and conditions prevail (temperature, humidity and the like) that can promote the growth of biofilms. For example, the solution containing chlorine dioxide can be fed to a water cycle of a bottle cleaning machine which supplies a pre-soak with water. Alternatively or in addition, the metering device can also be connected to spray nozzles or atomizers which are each arranged and configured to introduce the solutions containing chlorine dioxides into the chlorine dioxide consumer. Such embodiments (see also FIG. 2) are particularly advantageous when large regions, such as tunnels in a pasteurizer, have to be acted upon in a large scale with the solution containing chlorine dioxide in order to remove biofilms, where completely filling the respective region of the machine with water or a solution containing chlorine dioxide is not possible.

Metering device 102 can furthermore be connected to a control unit 180 which controls the metering device, in particular the output of solution containing chlorine dioxide by metering device 102 to the chlorine dioxide consumer(s). For this purpose, the control unit, which is designed, for example, as a computer or other processing unit for controlling systems, can also be connected to the chlorine dioxide consumers and/or chlorine dioxide producer 101. Control unit 180 can receive information from the chlorine dioxide consumers, for example, relating to a pH value within the chlorine dioxide consumer or a concentration of chlorine dioxide in the chlorine dioxide consumer. This data can then be used by control unit 180 for controlling the output of solution containing chlorine dioxide to the respective chlorine dioxide consumers in dependence of the pH value or the concentration of chlorine dioxide.

If, for example, a constant concentration of chlorine dioxide is desired in one region of a pasteurizer, then a sensor for detecting the concentration of chlorine dioxide in the chlorine dioxide consumer can then pass a signal that is indicative of the concentration to control unit 180, as is also explained in FIG. 2, which then determines by comparison of the measured concentration with a target value whether a solution containing chlorine dioxide must be added and, if so, how much of this solution must be added. This occurs when the chlorine dioxide concentration is below the desired target value. If the concentration is higher than the desired target value, then the control unit can determine that metering device 102 is, for example, to prevent the output of solution containing chlorine dioxide to the respective chlorine dioxide consumer until the concentration again drops to or below the desired target value.

Chlorine dioxide producer 101 can in principle be designed as a chemical reactor or other device which is suitable for producing chlorine dioxide using sodium chlorite and sulfuric acid in accordance with the following reaction equation

5 NaClO₂+2 H₂SO₄→4 ClO₂+NaCl+2 H₂O+2 Na₂SO₄.

The respective reaction takes place in an aqueous solution, i.e. in particular in the presence of water and the OH⁻ and H⁺ ions contained in neutral water. For the sake of clarity and since they do not take part in the reaction, they are not shown in the above equation.

It can be provided in one embodiment that the chlorine dioxide producer first produces a respective solution containing chlorine dioxide with the residues of the reaction (i.e. sodium chloride and sodium sulfate) having a chlorine dioxide concentration of 20,000 ppm. Other concentrations of 15,000 to 25,000 or in particular 17500 to 22500 ppm are also possible there. Such a concentration can be created without too much effort with regard to the reaction parameters. In particular, it can be provided that the solution containing chlorine dioxide produced in this manner is diluted with water directly, for example, still in chlorine dioxide producer 101 so that the concentration of chlorine dioxide in the aqueous solution (also referred to as the first dilution stage) drops to 2000 to 4000, preferably 2500 to 3500, in particular 3000 ppm. An aqueous solution containing chlorine dioxide at this concentration of chlorine dioxide is stable over a certain period of time and can therefore advantageously be stored if the solution containing chlorine dioxide produced is not immediately removed by the metering device.

In one embodiment it can also be provided for this purpose that a buffer tank 111 is provided into which the solution from chlorine dioxide producer 101, which has already been diluted to about 3000 ppm of chlorine dioxide concentration, is passed. This can be done, for example, via line 141 which leads from chlorine dioxide producer 101 into buffer tank 111. The solution containing chlorine dioxide can be stored there until it is used by metering device 102 that is connected to buffer tank 111. It can be connected to buffer tank 111 via line 142 and the solution containing chlorine dioxide from the buffer tank.

The use of a still relatively highly concentrated solution containing chlorine dioxide with a chlorine dioxide concentration of 3000 ppm and a respective concentration of the residues of sodium chloride and sodium sulfate in this solution can prove to be disadvantageous for some applications so that further dilution to a concentration in the now second dilution stage of 10 to 1000 ppm of chlorine dioxide in the aqueous solution is preferably performed either already in buffer tank 111 or by metering device 102.

Embodiments are particularly advantageous there in which a “standard solution” which has a certain predetermined concentration of chlorine dioxide, for example 3000 ppm or even 1000 ppm, is held available in buffer tank 111. In this case, metering device 102 can comprise a water tank or it can be associated with one such so that the metering device can further dilute a quantity of solution containing chlorine dioxide removed from buffer tank 111 by adding or mixing with water from the associated water tank until the desired concentration has been reached. This is particularly advantageous since the metering device can then remove the standard solution held available in buffer tank 111 for each chlorine dioxide consumer and in dependence of, for example, the required amount of chlorine dioxide, and dilute it to the appropriate concentration by adding water so that a suitable solution enriched with chlorine dioxide can be provided for any purpose in real time by metering device 102. It is provided that the standard solution in the buffer tank has a concentration that is higher than the concentration required in each chlorine dioxide consumer.

In particular the interaction with the control unit can be advantageous there. It can be provided that the control unit instructs the metering device to dose a solution containing chlorine dioxide in surges in dependence of a measured pH value or a concentration of chlorine dioxide measured in one of the chlorine dioxide consumers. In this case, it can be provided that the same volume quantity (for example 10 liters) of the solution containing chlorine dioxide is always fed to the respective chlorine dioxide consumer with such surge dosing. In order to ensure that also the appropriate quantity of chlorine dioxide is fed, standard solution 111 from the buffer tank can be diluted with water in such a way that the resulting quantity of solution containing chlorine dioxide to be fed to the chlorine dioxide consumer contains exactly the quantity of chlorine dioxide that is necessary to adjust the concentration of chlorine dioxide in the chlorine dioxide consumer to the desired value.

However, embodiments are also conceivable in which a buffer tank 111 is not provided. In such a case, the control unit can be connected not only to the metering device and the chlorine dioxide consumers, but also to chlorine dioxide producer 102. 0} If it is determined, for example, on the basis of a measured value of the concentration of chlorine dioxide in a chlorine dioxide consumer (for example 112) that solution containing chlorine dioxide must be fed to chlorine dioxide consumer 112, then the control unit can first instruct chlorine dioxide producer 101 to produce the solution containing chlorine dioxide. This can be either a solution containing chlorine dioxide corresponding to the “standard solution” described above or the chlorine dioxide producer can reduce the concentration of chlorine dioxide in the solution produced by adding water to the concentration in accordance with the second dilution stage, i.e. 10 to 1000 ppm, in particular 50 to 300 ppm. It can there be provided that the chlorine dioxide producer only produces as much solution containing chlorine dioxide as is to be delivered to the chlorine dioxide consumer by the metering device. This embodiment is preferred when the total consumption of solution containing chlorine dioxide is only relatively low and excess solution containing chlorine dioxide produced would have to be stored for a long period of time before it was used in a chlorine dioxide consumer. Due to the volatility of chlorine dioxide, the solution containing chlorine dioxides can finally be stored in part only with great effort while constantly being cooled and protected from light.

However, if the consumption of solution containing chlorine dioxide in the container treatment plant is comparatively high (for example several tens of liters per hour), then the embodiment first described, in which the chlorine dioxide producer produces solution containing chlorine dioxide continuously and feeds it to buffer tank 111, can be advantageous because direct addition of solution containing chlorine dioxide by the metering device to the corresponding chlorine dioxide consumers is always possible and there is no “waiting time” during which the chlorine dioxide producer first has to produce the solution containing chlorine dioxide.

The embodiment of a chlorine dioxide consumer in the form of a pasteurizer described in FIG. 2 can be combined with any of the embodiments just described with regard to the metering device, chlorine dioxide producer 101, and in particular buffer tank 111 from FIG. 1 where this appears appropriate.

The pasteurizer shown in FIG. 2 as one of chlorine dioxide consumers 112 is provided as a tunnel pasteurizer and for this purpose comprises a tunnel 220 through which the bottles are transported in the form of a disorderly mass flow. Pasteurizers of this type are typically several meters long and transportation through them takes place only slowly so that the dwelling time of bottles 230 in the pasteurizer can be from several minutes to a few hours. The pasteurizer is typically divided into three regions. In a region 221 arranged first in the transport direction, the heating region, bottles 230, which at this point in time have already been filled with a product and sealed, are heated. This is typically done by being acted upon with sprayed hot water from a suitable device 251, for example, an array of nozzles or atomizers.

The bottles thus heated are then taken to region 222 known as the pasteurization zone. In this region, a heating medium, in particular water, is again applied by a suitable device for application 252 which can be designed similarly to devices 251. The temperature in this region is kept high such that the product is pasteurized. Typical temperatures there are over 50° C., in some cases even over 80° C.

In the direction of transport of bottles 230, pasteurization zone 222 is followed by cooling zone 223. Bottles 230 are therein acted upon again with a medium using a suitable device 253 (configured similarly to devices 251) so that they are cooled to a lower temperature than in pasteurization zone 222. Water can be used again for this purpose.

Cooling zone 223 and heating zone 221 are advantageously, but not necessarily connected to one another in the form of a recuperation circuit. For example, the cold water that is used to cool bottles 230 in cooling zone 223 can be fed via a suitable line 270 to heating zone 221, in particular to device 251 in the heating zone in order to convert the quantity of heat absorbed by the initially cold water from the warm bottles of cooling zone 223 to heat the cold bottles in region 221 This can save energy. Conversely, a line 280 can be provided which feeds the water was used in heating zone 221 and has now cooled to cooling zone 223 in order to cool the bottles that have arrived there.

It is provided in this embodiment that metering device 102 is connected to at least one of zones 221, 222 or 223 via suitable lines 211 to 213. These can be lines equipped with pumps which in particular can distribute the solution containing chlorine dioxide through device for application 251 to 253 in the respective regions. Since the temperature in pasteurization zone 222 is typically above 50°, feeding solution containing chlorine dioxide during operation of the pasteurizer can be dispensed with there. Nevertheless, a line 212 to device for application 252 can be provided in this region for achieve an application of solution containing chlorine dioxide during a downtime of the pasteurizer during which pasteurization zone 222 is also cooled

Heating zone 221 and cooling zone 223 can be acted upon with solution containing chlorine dioxide either separately or independently of one another. A sensor 261 or 263 can be provided in each of these zones and be configured to measure a pH value of the water used and/or to measure a concentration of chlorine dioxide in the water used. Depending on the measured value, the metering device can then feed solution containing chlorine dioxide to the device for application 251 or 253 of respective zone 221 or 223. This embodiment is particularly advantageous when zones 221 and 223 do not form a closed system for recuperation.

In the event that these zones form a respective system for the recuperation of the heat given off by the bottles after having left pasteurization zone 222, as described above, these zones are advantageously connected to one another via lines 270 and 280. In this case, it can be provided that, if only one of sensors 261 or 263 outputs a measured value for the pH value or the concentration of chlorine dioxide which is below a predetermined limit value, then metering device 102 feeds solution containing chlorine dioxide to device 253 for acing upon the bottles in cooling zone 223. The chlorine dioxide contained in this solution is then not only distributed in region 223, but also additionally introduced into region 221 via line 270. Alternatively or in addition, the introduction can also take place via line 280 from region 221 to region 223. This ensures that the desired value for chlorine dioxide is obtained in both regions.

The embodiment just described for either regions 221 and 223 coupled in the form of a recuperation circuit or for regions 221 and 223 operated in isolation can be used in particular where a substantially continuous feed of solution containing chlorine dioxide is to be used. Sensors 261 and 263 then measure the pH value and/or the concentration of chlorine dioxide continuously or at certain time intervals (several minutes) with which the control unit described in FIG. 1 then causes the metering device and possibly the chlorine dioxide producer to output a solution containing chlorine dioxide as described above.

If regions 221 and 223 are configured as a closed recuperation system, then it can be particularly advantageous to have a sensor 271 be arranged in line 270 which measures the chlorine dioxide concentration in the solution passed through line 270. Sensors 261 and 263 can then be obsolete. Alternatively or in addition, a sensor 281 can also be provided there in line 280 which measures the chlorine dioxide concentration of the solution passed through this line.

It is understood that in such a closed recuperation system, the water cooling down in heating zone 221, which gives off heat to the bottles to be heated, can of course be passed into region 223 after the bottles have been heated in order to serve as a cooling medium there for the bottles and absorb heat given off by them. Any heat losses that occur can be compensated for by a heating device in the region of line 270 in that the water transported therein is always kept at a constant temperature.

Alternatively or in addition to the embodiments just described, it can also be provided that metering device 102 does not introduce any solution containing chlorine dioxide into the pasteurizer when the pasteurizer is in operation. In this case, it can be provided that it is verified while the machine is at a standstill (such as during maintenance work) whether the removal of biofilms is necessary. In such a case, metering device 102 can then be controlled accordingly to introduce a more highly concentrated solution containing chlorine dioxide into the pasteurizer, in particular, into regions 221 to 223, while the pasteurizer is at a standstill. A more highly concentrated solution containing chlorine dioxide with, for example, a concentration of 2 mg chlorine dioxide per liter can also successfully remove biofilms that have already formed, whereas with the continuous operation described above and the continuous feed of solution containing chlorine dioxide, a lower concentration of 1 mg per liter or 0.1 mg per liter can be sufficient to prevent the formation of biofilms as completely as possible. Combinations thereof are also conceivable so that solution containing chlorine dioxide is continuously added during continuous operation so that a certain concentration of chlorine dioxide can be found permanently in the water used in the pasteurizer in zones 221 and 223 in order to at least in part suppress the formation of biofilms. During maintenance, a highly dosed solution containing chlorine dioxide can additionally be introduced.

While the description of FIG. 2 was geared toward the components or at least regions 221-223 being acted upon in order to prevent the formation of biofilms or undesired deposits, it is understood that the water used in the pasteurizer (process water) can also be disinfected/cleaned in this way by the introduction of the solution containing chlorine dioxide. Microbiotic residues can thus already be killed in the process water and therefore before they are deposited on components of the container treatment machine. This naturally also applies to all of the embodiments described with reference to FIG. 1, so that not only disinfection or cleaning of the individual components of the respective machine takes place by the metering device introducing the solution containing chlorine dioxide into a container treatment machine of the container treatment plant, but the process water is simultaneously, additionally or alternatively cleaned/disinfected.

As already stated above, the configurations are not restricted to the use of pasteurizers in combination with the metering device. Other embodiments with tunnel heat exchangers, heaters, in particular tunnel heaters, or cooling towers required for cooling the container treatment plants are also conceivable.

1. Container treatment plant for treating containers, such as bottles, in a beverage processing industry comprising a chlorine dioxide producer, a chlorine dioxide consumer comprising at least one container treatment machine, and a metering device that can introduce a solution containing chlorine dioxide produced in said chlorine dioxide producer into said chlorine dioxide consumer, where said chlorine dioxide producer is configured to produce chlorine dioxide through a reaction of sodium chlorite and sulfuric acid.

2. Container treatment plant according to claim 1, where said chlorine dioxide consumer comprises a cooling tower and/or a tunnel heat exchanger and/or a tunnel heater.

3. Container treatment plant according to claim 1, further comprising a buffer tank for receiving solution produced in said chlorine dioxide producer and for delivering the solution received to said metering device.

4. Container treatment plant according to claim 1, where a delivery rate of said metering device can be controlled by a control unit.

5. Container treatment plant according to claim 1, where said metering device can introduce the solution into a wet region of said chlorine dioxide consumer.

6. Container treatment plant according to claim 5, further comprising a pH value sensor for measuring a pH value in said wet region and/or a chlorine dioxide sensor for measuring a concentration of chlorine dioxide in said chlorine dioxide consumer.

7. Method for disinfecting process water and/or a component of a container treatment plant comprising a chlorine dioxide producer, a chlorine dioxide consumer comprising at least one container treatment machine and a metering device, where said metering device introduces a solution containing chlorine dioxide into said chlorine dioxide consumer so that process water in said chlorine dioxide consumer and/or a component of said chlorine dioxide consumer is acted upon with the solution, where the solution is produced in said chlorine dioxide producer through a reaction of sodium chlorite and sulfuric acid.

8. The method according to claim 7, where an introduction of the solution by said metering device is controlled with an aid of a control unit so that the introduction takes place continuously or as surge dosing.

9. The method according to claim 8, where a pH value is measured in a wet region of said chlorine dioxide consumer and/or a concentration of chlorine dioxide in said chlorine dioxide consumer is measured and the introduction of the solution into said wet region is controlled in dependence of the pH value and/or the concentration.

10. The method according to claim 7, where said chlorine dioxide consumer comprises a pasteurizer with at least one heating zone, a pasteurization zone and a cooling zone, where said metering device adds the solution to said pasteurizer as follows:
in all zones at same or different concentrations; or
only in said heating zone or only in said cooling zone or only in said pasteurization zone; and/or
in at least one zone during a standstill of said pasteurizer; and/or
in a measured value-controlled or throughput-controlled manner.

11. The method according to claim 9, where a concentration of chlorine dioxide in the solution produced by said chlorine dioxide producer is 15,000 to 25,000 ppm and/or a concentration of the solution that is introduced into said chlorine dioxide consumer by said metering device is diluted in a first dilution stage to a concentration of 2000 to 4000 ppm, and/or a concentration of the solution that is introduced into said chlorine dioxide consumer by said metering device is diluted in a second dilution stage to 10 up to 1000 ppm and/or a chlorine dioxide concentration in the process water of said consumer is specified at 0.01 to 10 ppm as a target value.

12. The method according to claim 9, where said chlorine dioxide producer continuously produces the solution through the reaction of sodium chlorite with sulfuric acid and feeds the solution produced to a buffer tank, where said metering device removes the solution from said buffer tank.

13. The method according to claim 12, where said metering device feeds the solution to several chlorine dioxide consumers and where said metering device sets a concentration of chlorine dioxide in the solution for each chlorine dioxide consumer independently by adding water to the solution before said metering device introduces the solution into said chloride dioxide consumer.

14. The method according to claim 9, where the introduction of the solution into said chlorine dioxide consumer is effected at a temperature T<50° C., at least in a region of said chlorine dioxide consumer into which the solution is introduced.

15. The method of claim 1, wherein the at least one container treatment machine is a pasteurizer.

16. The method of claim 15, wherein the pasteurizer is a tunnel pasteurizer.

17. The method of claim 7, wherein the at least one container treatment machine is a pasteurizer.

18. The method of claim 17, wherein the pasteurizer is a tunnel pasteurizer.

19. The method of claim 11, wherein the concentration of chlorine dioxide in the solution produced by said chlorine dioxide producer is 17500 to 22,500 ppm.

20. The method of claim 11, wherein the solution that is introduced into said chlorine dioxide consumer by said metering device is diluted in the first dilution stage to a concentration of 2500 to 3500 ppm.
